(19) United States
(12) Patent Application Publication
(10) Pub. No.: US 2018/0028634 A1
(43) Pub. Date: Feb. 1, 2018

(54) METHOD FOR PROLONGING AND ENHANCING ANTI-TUMOR VACCINE RESPONSE

(71) Applicant: Lan Bo Chen, Lexington, MA (US)

(72) Inventor: Lan Bo Chen, Lexington, MA (US)

(21) Appl. No.: 15/664,197

(22) Filed: Jul. 31, 2017

Related U.S. Application Data
(60) Provisional application No. 62/368,674, filed on Jul. 29, 2016.

(51) Int. Cl. A61K 39/00; A61K 39/385; C07K 16/30; A61K 39/395

(52) U.S. Cl. CPC A61K 39/0011; A61K 39/39558; A61K 39/385; C07K 16/3076; A61K 2039/505; A61K 2039/5158; A61K 2039/55511; A61K 2039/6037

(57) ABSTRACT

A method for treating a tumor includes administering (i) a vaccine that contains stage-specific embryonic antigen 4 conjugated to a carrier, (ii) an antibody that binds specifically to SSEA-4, and (iii) immune cells expressing a chimeric antigen receptor that specifically binds to SSEA-4. The tumor, which expresses SSEA-4, can be a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

### CROSS-REFERENCE TO RELATED APPLICATIONS

The present application claims priority to Provisional Application No. 62/368,674, filed on Jul. 29, 2016. The content of this prior application is hereby incorporated by reference in its entirety.

### BACKGROUND

The goal of immunotherapy for cancer is to increase the strength of a patient's own immune responses against tumors. Immunotherapy can be implemented via several diverse treatment modalities.

For example, therapeutic antibodies have been developed that specifically bind to carbohydrate antigens on tumor cells, resulting in death of the cells via recruitment and stimulation of T cells. These carbohydrate antigens, e.g., stage-specific embryonic and stage-specific embryonic antigen 4 (“SSEA4”), are expressed in a wide variety of tumor is types and are not expressed in most adult tissues. See, e.g., Lee et al. 2014, J. Am. Chem. Soc. 136:16844-16853 (“Lee et al.”). The effectiveness of therapeutic antibodies is often limited due to suppression of T cell activity by tumor cells.

In another example, anti-cancer vaccines based on SSEA4 have been developed by fusion of this carbohydrate antigen to a carrier protein and co-administering it with glycolipid adjuvants. See Lee et al. Yet, carbohydrate antigens typically do not stimulate a robust immune response.

A further example of immunotherapy focuses on chimeric antigen receptors (“CARs”) developed to program T cells, NK cells, and NKT cells to attack tumor cells bearing a particular tumor antigen. A CAR contains an extracellular domain that binds to the tumor antigen and one or more intracellular domains that provide both primary and co-stimulatory signals to the T cells, NK cells, and NKT cells. These cells are isolated from a patient, engineered in vitro to express a CAR having an extracellular domain of choice, and infused back into the patient. Infusing such engineered cells is not without serious side-effects, among them being graft-versus-host disease and cytokine release syndrome.

There is a need for immunotherapy methods for treating tumors that combine the advantages of existing therapies while avoiding their drawbacks.

### SUMMARY

To meet this need, a method is provided for treating a tumor. The method includes administering to a subject having a tumor that expresses stage-specific embryonic antigen 4 (“SSEA4”) (i) a vaccine that contains SSEA4 conjugated to a carrier, (ii) an antibody that binds specifically to SSEA-4 (“anti-SSEA4 Ab”), and (iii) immune cells expressing a chimeric antigen receptor (“CAR”) that specifically binds to SSEA-4.

The details of one or more embodiments of the invention are set forth in the drawing and description below. Other features, objects, and advantages of the invention will be apparent from the description and from the claims.

Importantly, all references cited herein are hereby incorporated by reference in their entirety.

### BRIEF DESCRIPTION OF THE DRAWING

The description below refers to the accompanying drawing.

FIG. 1 shows the chemical structures of glycolipid adjuvants α-galactosylceramide (“αGalCer” or “C1”), derivatives of αGalCer, α-glucosylceramide (“αGlcCer”), and derivatives of αGlcCer.

### DETAILED DESCRIPTION

As mentioned above, the method of the invention includes administering a vaccine that contains SSEA4 conjugated to a carrier. The carrier can be, but is not limited to, bovine serum albumin (BSA), diphtheria toxoid cross-reactive material 197 (DT), bamboo mosaic virus (BMV), keyhole limpet hemocyanin (KLH), and tetanoid toxin (TT). SSEA4 can be conjugated to a carrier following the procedures set forth in U.S. Pat. No. 9,028,836.

In one embodiment, the SSEA4 is modified with an azido moiety. The azido moiety can be at the reducing end of SSEA4 or preferably at its non-reducing end. Azido modification of SSEA4 can be carried out as set forth in Lee et al.

A preferred vaccine contains SSEA4 conjugated to DT and azido-modified at its non-reducing end.

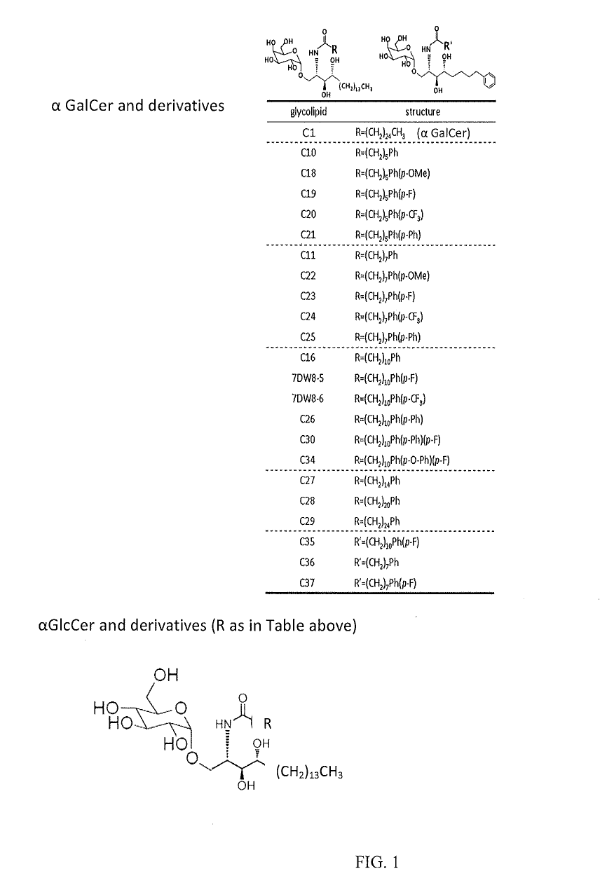

The vaccine described, supra, can also include an adjuvant. The adjuvant can be a glycolipid. Examples of a glycolipid include α-galactosylceramide and derivatives of α-galactosylceramide, such as those shown in FIG. 1 and described in International Application Publication 2008/128207 and in U.S. Pat. No. 9,028,836. In a preferred vaccine, the glycolipid is α-galactosylceramide C34.

The glycolipid can also be α-glucosylceramide or derivatives of α-glucosyl ceramide. The structure of α-glucosylceramide is shown in FIG. 1 as well. Derivatives of α-glucosylceramide can be the same derivatives as those shown for α-galactosylceramide. α-glucosylceramide C34 is among the preferred derivatives of α-glucosylceramide.

In a particular embodiment, the vaccine includes the adjuvant α-galactosylceramide C34 or α-glucosylceramide C34 and the antigen/carrier conjugate azido-SSEA4/DT.

The method of the invention also includes administering an anti-SSEA4 Ab. For example, the anti-SSEA4 Ab can be a fully humanized monoclonal IgG antibody. Alternatively, it can be a chimeric antibody including a single chain Fv (“scFv”) that specifically binds to SSEA4. Exemplary anti-SSEA4 Abs are described in US Patent Application Publication 2016/0102151.

In certain embodiments, the anti-SSEA4 Ab is linked to a cytokine, a cytotoxic agent, a modified immunoglobulin Fc domain, anti-CD3, or anti-CD16.

A cytokine can be fused to the anti-SSEA4 Ab as part of a fusion protein. See Kiefer et al. 2016, Immunol. Revs. 270:178-192. In another example, the cytokine is linked to the anti-SSEA4 Ab via cross-links between lysine residues. Exemplary suitable cytokines include G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

Exemplary cytotoxic agents are Diphtheria toxin, Pseudomonas exotoxin A (“PE38”), doxorubicin, methotrexate, an auristatin, a maytansine, a calicheamicin, a duocarmycin, a pyrrolobenzodiazepine dimer, and 7-ethyl-10-hydroxy-camptothecin. Suitable cytotoxic agents are described in Peters et al. 2015, Biosci. Rep. 35:1-20 (“Peters et al”); Bouchard et al. 2014, Bioorg. Med. Chem. Lett. 24:5357-5363; Panowski et al. 2014, mAbs 6:34-45; and Mazor et al. 2016, Immunol. Revs. 270:152-164.

The cytotoxic agent can be linked to the anti-SSEA4 Ab via a linker. In an embodiment, the linker is cleavable such that, upon internalization of the bifunctional agent by a tumor cell, the cytotoxic agent is cleaved from the binding domain. Examples of a cleavable linker include, but are not limited to, acid-labile small organic molecules (e.g., hydrazone), protease cleavable peptides (e.g., valine-citrulline dipeptide), and disulfide bonds. In another embodiment, the linker is not cleavable. In this case, the cytotoxic agent is released upon degradation of the anti-SSEA4 Ab linked to it. Additional examples of linkers are described in Peters et al.

If the cytotoxic agent is a protein, it can be linked to the anti-SSEA4 Ab via a peptide bond, e.g., as part of a fusion protein. In a particular example, PE38 can be fused to the C-terminus of a V_{L} chain of an anti-SSEA4 monoclonal antibody.

In a particular embodiment, the anti-SSEA4 Ab is an anti-SSEA4 antibody fragment, e.g., an anti-SSEA4 scFv, linked to a modified immunoglobulin Fc domain. For example, the Fc domain can be modified such that it specifically targets the FcγRIIa receptor, the FcγRIIIa receptor, or the FcRn receptor, as compared to an unmodified Fc domain. Targeting the FcγRIIa or FcγRIIIa receptor leads to an increased cytotoxic immune response. On the other hand, targeting the FcRn receptor increases the half-life of the anti-SSEA4 antibody. Modifications to the Fc domain that increase its affinity for the FcγRIIa receptor, the FcγRIIIa receptor, or the FcRn receptor are described in Moore et al. 2010, mAbs 2:181-189 and Lobner et al. 2016, Immunol. Revs. 270:113-131.

In another embodiment, the anti-SSEA4 Ab is linked to an anti-CD3 molecule. The anti-CD3 molecule activates T cells localized to tumor cells via the anti-SSEA4 Ab. An exemplary anti-CD3 molecule is an antibody fragment. The anti-CD3 molecule can specifically bind to CD3ε. Further, a scFv that specifically binds to SSEA4 can be fused to another scFv that specifically binds to CD3.

In still another embodiment, the anti-SSEA4 Ab is linked to an anti-CD16 molecule. The anti-CD16 molecule activates NK cells localized to tumor cells via the anti-SSEA4 antibody. Like the anti-CD3 molecule described in the preceding paragraph, the anti-CD16 molecule can be an antibody fragment that binds specifically to CD16. Exemplary constructs are an anti-SSEA4/anti-CD16 chimeric antibody and a scFv that specifically binds to SSEA4 fused to another scFv that specifically binds to CD16.

As mentioned, supra, the method of the invention also requires administering immune cells bearing a CAR that specifically binds to SSEA4.

The CAR includes a scFv that binds specifically to SSEA4. The scFv that specifically binds to SSEA4 can be, e.g., any of those exemplified in US Patent Application Publication 2016/0102151.

In addition to the scFv, the CAR also contains an endodomain from CD3ζ or FcεRIγ. The endodomain contains one or more immunoreceptor tyrosine-based activating motifs (“ITAM”).

The CAR further includes a hinge/spacer region and a transmembrane region between the scFv and the endodomain.

Exemplary sequences that can be used as a hinge/spacer region are derived from the hinge region of, e.g., IgG1, IgG4, and IgD. Alternatively, it can be derived from CD8. See, e.g., Dai et al. 2016, J. Natl. Cancer Inst. 108:1-14 (“Dai et al.”) and Shirasu et al., 2012, Anticancer Res. 32:2377-2384 (“Shirasu et al.”).

Exemplary transmembrane regions that can be included in the CAR are derived from CD3, CD4, CD8, or CD28. See Dai et al. and Shirasu et al.

Optionally, the CAR also contains a second endodomain in addition to the endodomain from CD3ζ or FcεRIγ. The second endodomain, e.g., from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, and PILR, like the first endodomain, contains one or more ITAM.

Furthermore, the CAR can contain a third endodomain, which also can be from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR. The third endodomain is different from the second endodomain.

In a specific embodiment, the CAR contains an anti-SSEA4 scFv fused to a spacer/hinge from CD8 that is fused to a transmembrane domain also from CD8 fused to the N-terminus of the endodomain from CD28, which in turn is fused to the N-terminus of the endodomain from CD137, which in turn is fused to the N-terminus of the endodomain from CD3ζ.

In the method of the invention, the subject can be administered with one of three types of CAR-bearing immune cells, i.e., T cells, NK cells, NKT cells, or a mixture thereof. The T cells, NK cells, or NKT cells express any of the CARs described above. In one embodiment, such cells are obtained by transducing T cells, NK cells, or NKT is cells in vitro with an expression vector encoding the CAR. The T cells, NK cells, or NKT cells to be transduced can be isolated from the subject.

The expression vector includes a promoter operably linked to a nucleic acid encoding the CAR. The promoter is active in T cells, NK cells, or NKT cells.

Exemplary CAR expression vectors based on lentiviral vectors or a gamma retroviral vectors are set forth in Dai et al.; Jin et al. 2016, EMBO Mol. Med. 8:702-711; Liechtenstein et al. 2013, Cancers 5:815-837; and Schonfeld et al. 2015, Mol. Therapy 23:330-338 (“Schonfeld”).

Such expression vectors are used for integrating the promoter/CAR-encoding nucleic acid into T cell, NK cell or NKT cell genomic DNA to produce stable expression of the CAR.

Alternatively, the expression vector contains sequences that facilitate transposon-mediated genomic integration of the promoter/CAR-encoding nucleic acid into T cells, NK cells, or NKT cells. Examples of these expression vectors are the so-called “PiggyBac” and “Sleeping Beauty” expression vectors. See Nakazawa et al. 2011, Mol. Ther. 19:2133-2143 and Sourindra et al. 2013, J. Immunotherapy 36:112-123.

T cells, NK cells, or NKT cells are isolated from a subject suffering from a tumor. Procedures for isolating these cells are known in the art. See, e.g., Kaiser et al. 2015, Cancer Gene Therapy 22:72-78 (“Kaiser et al.”).

Established NK cell lines can also be used in the method instead of NK cells isolated from a subject. See, e.g., Schonfeld.

Expression vectors are transduced into T cells, NK cells, or NKT cells by, e.g., electroporation, lipofection, lentiviral infection, and gamma retrovirus infection.

The transduced cells are expanded in vitro, using methods known in the art. See Kaiser et al.

Finally, the expanded T cells, NK cells, or NKT cells are administered by infusion in one batch or in two or more batches into the subject having a tumor.

In one embodiment, the method of the invention includes a preconditioning step that is performed prior to the just-mentioned administering step. The preconditioning step is accomplished by treating the subject with a drug that induces lymphodepletion. is Examples of these drugs include cyclophosphamide and fludarabine. Additional drug examples can be found in Dai et al. and Han et al. 2013, J. Hematol. Oncol. 6:47-53.

To carry out the tumor treatment method set forth above, the subject to be treated can be administered simultaneously with the vaccine, the anti-SSEA4 Ab, and the immune cells expressing a CAR that binds to SSEA4.

In a preferred alternative, the vaccine, the anti-SSEA4 Ab, and the immune cells are administered separately. For example, the vaccine can be administered every week or every other week for two to three months, followed by the anti-SSEA4 Ab again every week or every other week for two to three months, further followed by administering T cells, NK cells, and/or NKT cells expressing the anti-SSEA4 CAR.

Preferably, the administration is via injection or infusion.

The method described above is effective for treating e.g., a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

Without further elaboration, it is believed that one skilled in the art can, based on the description above, utilize the present invention to its fullest extent.

The following references, some cited supra, can be used to better understand the background of the application:

Abate-Daga et al. 2016, Mol. Ther. Oncolytics 3:1-7

Berti et al. 2013, ACS Chemical Biol. 8:1653-1663

Bouchard et al. 2014, Bioorg. Med. Chem. Lett. 24:5357-5363

Becker et al. 2010, J. Immunol. 184:6822-6832

Carreno et al., Clin. and Translational Immunol. 5(e69):1-9.

Chen et al., J. Clin. Invest. 125:3384-3391

Chen et al. 2013, Adv. Drug Deliv. Rev. 65:1357-1369

Cheung et al. 2016, Proc. Natl. Acad. Sci. 113:960-965

Curran et al. 2012, J. Gene Med. 14:405-415

Dai et al. 2016, J. Natl. Cancer Inst. 108:1-14

Danishefsky et al. 2015, Acc. Chem. Res. 48:643-652

DiLillo et al. 2015, Cancer Immunol. Res. 3:704-713

Guest et al., 2005, J. Immunother. 28:203-211

Han et al. 2013, J. Hematol. Oncol. 6:47-53

Heczey et al. 2014, Blood 124:2824-2833

Huang et al. 2013, Proc. Natl. Acad. Sci. 110:2517-2522

James et al. 2008, J. Immunol. 180:7028-7038

Jiang et al. 1999, Infection and Immmunity 67:2996-3001

Kaiser et al. 2015, Cancer Gene Therapy 22:72-78

Keir et al., Annu. Rev. Immunol. 26:677-704

Kiefer et al. 2016, Immunol. Revs. 270:178-192

Lawson 2012, Immunology 137:20-27

Lee et al. 2014, J. Am. Canc. Soc. 136:16844-16853

Lieschke et al. 1997, Nature Biotech. 15:35-40

Liu et al. 2008, Eur. J. Immunol. 38:1012-1023

Lo et al. 1998, Protein Engineering 11:495-500

Lou et al., Proc. Natl. Acad. Sci. 111:2482-2487

Lobner et al. 2016, Immunol. Revs. 270:113-131

Mazor et al. 2016, Immunol. Revs. 270:152-164

Moore et al. 2010, mAbs 2:181-189

Moritz et al. 1995 Gene Therapy 2:539-546

Nakazawa et al. 2011, Mol. Ther. 19:2133-2143

Panowski et al. 2014, mAbs 6:34-45

Pauken et al., J. Immunol. 194:3551-3555

Pegram et al. 2011, Immunol. Cell Biol. 89:216-224

Peters et al. 2015, Biosci. Rep. 35:1-20

Rajagopalan et al. 2005, J. Exp. Med. 201:1025-1029

Rezvani et al. 2015, Front. Immunol. 17 November

Rodgers et al. 2016, Proc. Natl. Acad. Sci. Jan. 12:E459-E468

Ruggeri et al. 2002, Science 295:2097-2100

Schonfeld et al. 2015, Mol. Therapy 23:330-338

Sharma et al. Science 348:56-61

Shirasu et al. 2012, Anticancer Res. 32:2377-2384

Sourindra et al., 2013, J. Immunotherapy 36:112-123

Thermo Scientific 2012, Crosslinking Technical Handbook

Zhu et al. 2007, J. Immunol. 178:5435-5442

The contents of the above references are hereby incorporated by reference in their entirety.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for treating a tumor, the method comprising administering to a subject having a tumor a vaccine that contains stage-specific embryonic antigen 4 (SSEA4) conjugated to a carrier, an antibody that binds specifically to SSEA-4 (anti-SSEA4 Ab), and immune cells expressing a chimeric antigen receptor (CAR) that specifically binds to SSEA-4, wherein the tumor expresses SSEA-4.

2. The method of claim 1, wherein the vaccine further contains α-galactosylceramide C34 or α-glucosylceramide C34 as an adjuvant.

3. The method of claim 2, wherein the immune cells are T cells, NK cells, NKT cells, or a mixture thereof and the CAR contains (i) a scFv that binds specifically to SSEA4 and (ii) a CD3ζ endodomain or an FcεRIγ endodomain.

4. The method of claim 3, wherein the CAR further contains a second endodomain from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR.

5. The method of claim 4, wherein the vaccine, the anti-SSEA4 Ab, and the immune cells are administered sequentially.

6. The method of claim 5, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

7. The method of claim 6, wherein the anti-SSEA4 Ab is linked to an agent selected from the group consisting of a cytokine, a cytotoxic agent, a modified immunoglobulin Fc domain, anti-CD3, and anti-CD16.

8. The method of claim 7, wherein the anti-SSEA4 Ab is linked to a cytokine selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

9. The method of claim 7, wherein the anti-SSEA4 Ab is linked to a cytotoxic agent selected from the group consisting of Diphtheria toxin, Pseudomonas exotoxin A, doxorubicin, methotrexate, an auristatin, a maytansine, a calicheamicin, a duocarmycin, a pyrrolobenzodiazepine dimer, and 7-ethyl-10-hydroxy-camptothecin.

10. The method of claim 7, wherein the anti-SSEA4 Ab is linked to a modified immunoglobulin Fc domain modified to target the FcγRIIa receptor, the FcγRIIIa receptor, or the FcRn receptor.

11. The method of claim 7, wherein the anti-SSEA4 Ab is linked to anti-CD3 or anti-CD16.

12. The method of claim 3, wherein the carrier is diphtheria toxoid cross-reactive material 197 and the SSEA4 has an azido group at the non-reducing end.

13. The method of claim 12, wherein the CAR further contains a second endodomain from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR.

14. The method of claim 13, wherein the vaccine, the anti-SSEA4 Ab, and the immune cells are administered sequentially.

15. The method of claim 14, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

16. The method of claim 15, wherein the anti-SSEA4 Ab is linked to an agent selected from the group consisting of a cytokine, a cytotoxic agent, a modified immunoglobulin Fc domain, anti-CD3, and anti-CD16.

17. The method of claim 16, wherein the anti-SSEA4 Ab is linked to a cytokine selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

18. The method of claim 16, wherein the anti-SSEA4 Ab is linked to a cytotoxic agent selected from the group consisting of Diphtheria toxin, Pseudomonas exotoxin A, doxorubicin, methotrexate, an auristatin, a maytansine, a calicheamicin, a duocarmycin, a pyrrolobenzodiazepine dimer, and 7-ethyl-10-hydroxy-camptothecin.

19. The method of claim 16, wherein the anti-SSEA4 Ab is linked to a modified immunoglobulin Fc domain modified to target the FcγRIIa receptor, the FcγRIIIa receptor, or the FcRn receptor.

20. The method of claim 16, wherein the anti-SSEA4 Ab is linked to anti-CD3 or anti-CD16.
